SEQUENCE LISTING (35 sequences)

SEQ ID NO: 1 - DNA, 2225 bp, Danio
ccttcccttctaagtctgacgtccttttaagagcttgtgcatgaaagcaaatttagagcttattactcatctttaacacccatacaaagtgatgattgccgtaccgtgatctcacacctttcacacctggtttatactatgatagttgtagacgattgcgtaatgctattaaatgcccatcagtgctggccgtgacacccaactgctgccatttcatgttgacttgcacgagaaatgggaaattgtctgactatgcagggtgtaatatgcgtgggaatatttatcagtggtcattaaatactatagtttacagttagagcaaagtgtgctgtatttttgtgtcagcttagctgctgtttttgtatgtaaagtaacaaatgacaaatactcaaactattgaaattaagtagtttttctcagaaatttactaagtactttaaaaatgtgtacttttactttcccttgagtacatttttagtgcagtattggtacttttatttcactactttccttcaacctgcagtcactactttattttttcttgtctttgtggattagtcaaatcagtcctgattcctgtccaatcaattcgcacatagaaggtaaatcacatcataatgcactacctcaagacatgggcaatttataattgcagcaaactgtttgccagcattaaaagaagatgtcaaaaatctttacacacattaacccagagactgcttagatgcatgtcactgatgagaagatgatggatgtttactgtatgatgaccgaaataactttaaacgcacacaagacggcacaagacgtcaacatggcgttaggttgacgttgtaccccaacgcagtggggacgttgcattttgtttagaaatgaaaattaggttgatgtcagaactcacgtcaggtcgatgtcaatgttcgatcatccaatcgaaaatcatatatcaatgtctaaggtggtaacagcttgatgtgttgtgggggttacccctatgacgtctatcagacgttggattatggttgccatacctgatgaataaatgtcattatttgacgttggtttaagatgttggttcgacattggattttggtcgctttccaacacaacctaaatccaccaaatattaacttcctatgacatcgttattggacgtcaaaataacaatatccttagatgctggctagactttgaatttaggtcaccacaacctatatttaacctaatattaacatcttatgatgttgtgtgcctgctgggcaataactaaatgcactacagaatgttacgtttaccacatgtaaattacatgtaaatgcatcagcttttcacagcataatactcactacttactactcttgagtacttttaaaaaagctacttttcactcatactttgagtaatatttacaactgatacttttactcgcactacatttttaggcgtgtattgatatttttactatgattttcagtactctttccactactgcagccctccccatacataatcgtatgtttacacatatggtggagtttagagccataaactacattagctttgttagccgctagcattactgtgcagaattgtgtgtgtgcacattttccaatatcaatacagaaggaaactgtgttccctgttcccttgtaaatctcaacaatgcaactgttcagctcagggggaaaaatgccctgccagatccaaacgctggcaaaagtgaatggaaaaaagcctttcattaatgtgaaagttgctgcgcgccccacccagataaaaagagcagaggttaacatgctctctacggctgtccagccaaccagatactgaggcagaaacacacccgctggcagatggtgagagctacactgtctttttcagagtttctactggaatgcctgtcctcaagtctcaagcctctccttgcattttctcattccacctggggcaaagccccaggctgggtgtgacaacatttatcttaccactttctctctgtacctatctaacaggtagggtgtgtgtgagagtgcgtatgtgtgcaagtgtgtgtgtgagagcagtcagctccaccccctcaagagtgtgtataaaattggtcagccagctgctgagagacacgcagagggactttgactctcctttgtgagcaacctcctccactcactcctctctcagagagcactctcgtacctccttctcagcaactcaaagacacaggcatc

SEQ ID NO: 2 - DNA, 540 bp, Ctenopharyngodon
atgaaaactcaaatgtggacgtatatgtttgtaatgtttttaactctgcagggtcaatgctctgcttgcgaatggctcggccgatacaggatgataagcaacgagtctttgagcctcctgaaggaaatgggtggaaaatatcctgagggtaccaaggtgtcatttccaggacgcctgtacaacatgatagacaatgccaaggtggaggaccaggtgaagtttcttgtcctgaccttagatcatatcatccgcctcatggatgccagagagcacatgaattcagtgcagtggaacctacagactgtagagcattttctaactgtcctgaacaggcagtcatctgatcttaaagaatgtgtggcccgataccagccatcacataaggagtcctacgagaaaaagataaacagacacttcaagattttaaagaagaatctaaagaaaaaagaatatagtgctcaagcatgggagcagatccggagagctgtgaaacatcaccttcagaggatggacatcatcgcaagcattgccaacagacga

SEQ ID NO: 3 - DNA, 27 bp, Artificial Sequence
  synthesized in laboratory
ctcgagatgaaaactcaaatgtggacg

SEQ ID NO: 4 - DNA, 26 bp, Artificial Sequence
  synthesized in laboratory
cctaggagcagacaaccgttacgaac

SEQ ID NO: 5 - DNA, 21 bp, Artificial Sequence
  synthesized in laboratory
ccttcccttctaagtctgacg

SEQ ID NO: 6 - DNA, 21 bp, Artificial Sequence
  synthesized in laboratory
gatgcctgtgtctttgagttg

SEQ ID NO: 7 - DNA, 24 bp, Artificial Sequence
  synthesized in laboratory
cagagggactttgactctcctttg

SEQ ID NO: 8 - DNA, 22 bp, Artificial Sequence
  synthesized in laboratory
atgatgatgatgatgatggtcg

SEQ ID NO: 9 - DNA, 24 bp, Artificial Sequence
  synthesized in laboratory
gaatgcctgtcctcaagtctcaag

SEQ ID NO: 10 - DNA, 22 bp, Artificial Sequence
  synthesized in laboratory
cgtcctggaaatgacaccttgg

SEQ ID NO: 11 - DNA, 21 bp, Artificial Sequence
  synthesized in laboratory
cgactgtgccttctagttgcc

SEQ ID NO: 12 - DNA, 22 bp, Artificial Sequence
  synthesized in laboratory
gcctgctattgtcttcccaatc

SEQ ID NO: 13 - DNA, 24 bp, Artificial Sequence
  synthesized in laboratory
cgaccatcatcatcatcatcattg

SEQ ID NO: 14 - DNA, 24 bp, Artificial Sequence
  synthesized in laboratory
cgatacaggatgataagcaacgag

SEQ ID NO: 15 - DNA, 21 bp, Artificial Sequence
  synthesized in laboratory
cagccatcacataaggagtcc

SEQ ID NO: 16 - DNA, 23 bp, Artificial Sequence
  synthesized in laboratory
ctgtatcacaattccagtgggtc

SEQ ID NO: 17 - DNA, 25 bp, Artificial Sequence
  synthesized in laboratory
ggcatttaatagcattacgcaatcg

SEQ ID NO: 18 - DNA, 21 bp, Artificial Sequence
  synthesized in laboratory
agaccagcaagacgaatacga

SEQ ID NO: 19 - DNA, 19 bp, Artificial Sequence
  synthesized in laboratory
tgttgacggcacctccatt

SEQ ID NO: 20 - DNA, 22 bp, Artificial Sequence
  synthesized in laboratory
gctggacatctcagaaccttac

SEQ ID NO: 21 - DNA, 20 bp, Artificial Sequence
  synthesized in laboratory
ctcctcctgctgctccttac

SEQ ID NO: 22 - DNA, 20 bp, Artificial Sequence
  synthesized in laboratory
gtggcacggctctgcaagtt

SEQ ID NO: 23 - DNA, 22 bp, Artificial Sequence
  synthesized in laboratory
caaccgaggcaccatcaaccat

SEQ ID NO: 24 - DNA, 21 bp, Artificial sequence
  synthesized in laboratory
tgcgacaacggctgctttgat

SEQ ID NO: 25 - DNA, 21 bp, Artificial sequence
  synthesized in laboratory
ttgcggacaaccaacggatgg

SEQ ID NO: 26 - DNA, 27 bp, Artificial sequence
  synthesized in laboratory
atgtaaacttctgacccactgggaatg

SEQ ID NO: 27 - DNA, 26 bp, Artificial sequence
  synthesized in laboratory
tggtgatcctaactgacctaagacag

SEQ ID NO: 28 - DNA, 23 bp, Artificial sequence
  synthesized in laboratory
cgacttcaactgagtcgacctcg

SEQ ID NO: 29 - DNA, 23 bp, Artificial sequence
  synthesized in laboratory
tcagacttagaagggaaggaagc

SEQ ID NO: 30 - DNA, 24 bp, Artificial sequence
  synthesized in laboratory
agtagatgtcctaactgacttgcc

SEQ ID NO: 31 - DNA, 24 bp, Artificial sequence
  synthesized in laboratory
atagtgagtcgtattacgcgcgct

SEQ ID NO: 32 - DNA, 16 bp, Artificial sequence
  synthesized in laboratory
  misc_feature (5)..(5): n is a, c, g, or t
  misc_feature (10)..(10): n is a, c, g, or t
tgwgnagwancasaga

SEQ ID NO: 33 - DNA, 16 bp, Artificial sequence
  synthesized in laboratory
  misc_feature (5)..(5): n is a, c, g, or t
  misc_feature (10)..(10): n is a, c, g, or t
  misc_feature (13)..(13): n is a, c, g, or t
wgcangawgnagnatg

SEQ ID NO: 34 - DNA, 16 bp, Artificial sequence
  synthesized in laboratory
  misc_feature (5)..(5): n is a, c, g, or t
  misc_feature (10)..(10): n is a, c, g, or t
  misc_feature (13)..(13): n is a, c, g, or t
wgcangawgnagnatg

SEQ ID NO: 35 - DNA, 16 bp, Artificial sequence
  synthesized in laboratory
  misc_feature (1)..(1): n is a, c, g, or t
  misc_feature (8)..(8): n is a, c, g, or t
ntcgtsgnatstwgaa

(19) United States
(12) Patent Application Publication
(10) Pub. No.: US 2021/0310023 A1
(43) Pub. Date: Oct. 7, 2021

(54) Method for preparing fish skin mucous gland bioreactor and application thereof

(71) Applicant: Institute of Hydrobiology, Chinese Academy of Sciences, Wuhan (CN)

(72) Inventors: Zongbin Cui, Wuhan (CN); Tong Zhou, Wuhan (CN); Bolan Zhou, Wuhan (CN); Yong Long, Wuhan (CN); Guili Song, Wuhan (CN); Qing Li, Wuhan (CN); Zuoyan Zhu, Wuhan (CN)

(21) Appl. No.: 16/905,930

(22) Filed: Jun. 19, 2020

(30) Foreign Application Priority Data
Sep. 25, 2019 (CN) 201910912452X

(51) Int. Cl. C12N 15/85; A01K 67/027

(52) U.S. Cl. CPC C12N 15/8509; A01K 67/0275; C12N 2800/106; C12N 2015/8518; A01K 2207/05; A01K 2227/40; A01K 2267/01; C12N 2830/008

(57) ABSTRACT

A method for preparing a fish skin mucous gland bioreactor and its application, including: identifying genes specifically expressed in fish skin mucinous gland cells, promoters and secreted protein signal peptides, constructing transgenic expression vectors that can specifically express endogenous or heterologous biologically active substances in fish skin and mucous gland cells, developing stable genetic and transgenic fish that secrete bioactive substances into fish mucus, and using bioactive substances secreted by mucus glands for animal and plant growth, stress resistance and disease resistance, human health care and disease prevention, and commercial enzymes. The fish skin mucous gland bioreactor developed by the invention has the characteristics of easy breeding and expansion, more skin mucus secretion, convenient mucus collection, and easy purification of bioactive substances, and can realize the large-scale production of fish skin mucous gland bioreactor and efficient application.

### CROSS REFERENCE OF RELATED APPLICATION

The present application claims priority under 35 U.S.C. 119(a-d) to CN 201910912452X, filed Sep. 25, 2019.

### BACKGROUND OF THE PRESENT INVENTION

### Field of Invention

The invention belongs to the technical field of animal bioreactor preparation, and particularly relates to a method for preparing a fish skin mucous gland bioreactor and an application thereof. The bioreactor can be used for preparing polypeptides, proteins, enzymes, vaccines and antibodies.

### Description of Related Arts

A bioreactor refers to the transfer of a DNA sequence encoding a biologically active substance into the genome of a microorganism, yeast, animal or plant cell or tissue, or a living animal or plant, making it a container for efficient expression of a corresponding biologically active polypeptide or protein molecule. The use of bioreactors to produce biologically active peptides, proteins, enzymes, vaccines, antibodies, etc. is one of the frontiers of applied research in genetic engineering technology.

Currently commonly used bioreactors mainly include bacteria, yeast, plant cells or tissues, cultured animal and plant cells, living animals and plants or tissues (such as animal mammary glands) and other types. They have their own advantages, but there are one or more defects: Proteins expressed in bacteria cannot be properly post-translationally modified. Proteins expressed in fungi are immunogenic, and the culture conditions of isolated animal cells are harsh and costly. Animal mammary gland bioreactors have long development cycles and low success rates. In addition, researchers have also tried to develop various types of bioreactors such as avian egg white, mammalian blood, bladder, and semen, but there are problems such as difficulty in transgene manipulation, low expression efficiency, or effective expression of products that can adversely affect animal health.

Fish as an animal bioreactor has unique advantages, such as the correct post-translational modification of the expressed proteins, low production costs, fast expansion, and mature transgenic technology. Most fish bioreactors use ovaries or testes as target tissues.

The Chinese invention patent with the publication number of CN102796763B discloses a carp testis bioreactor, which uses the specific expression properties of fish testis genes to construct a safe, efficient and sustainable bioreactor.

However, the current existing technology has only progressed to the use of fish's reproductive system as a bioreactor. Whether other systems can still be used as bioreactors and what beneficial effects have yet to be studied by researchers.

### SUMMARY OF THE PRESENT INVENTION

In view of the problems existing in the prior art, the present invention provides a method for preparing a fish skin mucous gland bioreactor and its application. Including: constructing gene expression vectors that can specifically express bioactive substances (peptides, proteins, enzymes, vaccines, antibodies, etc.) in fish skin mucus cells, develop transgenic fish (such as loach, catfishes etc.) that stably express and secrete bioactive substances), isolation and purification of biologically active substances in mucus, and biological activity test. The fish skin mucous gland bioreactor prepared by the method of the invention can efficiently and cost-effectively produce various biologically active substances (polypeptides, proteins, industrial enzymes, vaccines, antibodies, etc.), and is applied to the growth, stress resistance and disease resistance of animals and plants, human health care and disease prevention, and commercial enzymes.

Technical solution of the present invention is achieved as follows. A method for preparing a fish skin mucous gland bioreactor, comprises steps of:

S1: obtaining specific expression promoters of fish skin and mucous gland cell;

S2: constructing a transgenic expression vector capable of specifically expressing the target biologically active substance, wherein the transgene expression vector comprises the specific expression promoter obtained in step S1; and

S3: digesting the transgenic expression vector obtained in step S2 and injecting into the fertilized eggs of the fish to obtain transgenic fish.

Furthermore, the step S1 obtaining specific expression promoters of fish skin and mucous gland cell comprises: using transcriptomics, comparing and analyzing gene expression profiles of different tissues or cells of fish, identifying genes specifically expressed in fish skin and mucus gland cells, and searching for specific expression promoters of genes; or using proteomics techniques, comparing and analyzing protein expression profiles of different tissues or cells, identifying genes specifically expressed in fish skin and mucous gland cells, and searching for promoters of specifically expressed genes; or searching published literature to find genes or promoters specifically expressed in fish skin and mucous gland cells.

Furthermore, searching for the promoter of a specifically expressed gene is to use a genomic sequence alignment method or a chromosome walking technique, combined with biological information analysis to find the promoter sequence upstream of the target gene.

Furthermore, the specific expression promoter of fish skin and mucous gland cells in step S1 is krt8 or lgals2b or rblec3 or glant8 or other mucous-specific promoters.

Furthermore, the target biologically active substance in step S2 is selected from any one of a polypeptide, a protein, an enzyme, a vaccine, or an antibody.

Furthermore, the step S3 is followed by a step of purifying the bioactive substance secreted by the skin mucous glands.

Furthermore, the step of purifying the biologically active substance includes purifying the biologically active substance with a resin.

Application of a method for preparing a fish skin mucous gland bioreactor as described above in the industrial production of biologically active substances.

The application of the bioactive substance produced by the method for preparing the fish skin mucous gland bioreactor in the production and preparation of polypeptides, proteins, enzymes, vaccines, antibodies or feed additives.

In summary, the advantages and positive effects of the present invention are:

Compared with fish eggs or testes, if fish skin mucous glands can be used as bioreactors, they have more prominent advantages. Fish skin mucus cells are widely distributed on the body surface, their secretion ability is strong, and they are not affected by age, season and gender restrictions. The secreted proteins are packed into the mucus vesicles, and the mucus can be released only when the mucus cells migrate to the surface of the body, and the expressed foreign protein will not adversely affect the cells and the body itself. The mucous glands of fish skin can release a large amount of mucus in a short period of time in the case of shock or environmental factors. Therefore, by stimulating the fish body, the mucus discharge time can be artificially controlled to facilitate the centralized collection of mucus and the extraction of bioactive substances in the mucus.

The invention proposes for the first time the concept of skin mucus glands of fish (such as loach, catfishes, etc.) as a bioreactor, and establishes a set of technical system for producing grass carp type I interferon (IFN1) by using transgenic loach skin mucus glands. The present invention is suitable for the production of any biologically active peptides, proteins, enzymes, vaccines, antibodies, etc.

The invention utilizes the obtained potential fish skin and mucinous gland cell specific promoter DNA sequences, clones them into a promoter activity test vector, microinjects them into single-cell fish fertilized eggs, and observes the promoter-driven fluorescence (such as EGFP, RFP, etc.) during embryonic development and tissue-specific expression of protein genes, thereby identifying skin and mucous gland cell-specific promoters.

The biologically active substance produced by the present invention should have the ability to secrete into the mucus. The biologically active substance needs to be possessed by itself or a suitable signal peptide must be added by molecular cloning. Firstly, use the SignalP 4.1 Server website to predict whether a biologically active substance has a signal peptide. If it does not have a signal peptide, you can use molecular cloning to add a suitable signal peptide to the N-terminus of the target gene to ensure the normal secretion of the biologically active substance. Secondly, the expression plasmid carrying the target gene is transfected into the cells, and the supernatant of the culture medium is collected, and the expression of the target protein is detected by Western blot, so as to detect the effect and efficiency of the biologically active substance signal peptide.

The present invention linearizes the transgene expression vector, and co-microinjects it with mature SB transposase mRNA synthesized in vitro into the fertilized eggs of fish (such as loach, catfishes etc.) to develop the PO generation of transgenic fish. At the juvenile stage, the genomic DNA is extracted from the tail fins, and PCR primers are designed to optimize the sensitivity and specificity of detecting the gene or tag sequence of the transposon system, skin-specific promoters, and bioactive substances. The optimized primer pairs and PCR method were used to obtain positive fish. F0 generation positive fish was crossed with wild type to obtain F1 generation positive fish, F1 generation positive fish was crossed with wild type to obtain F2 generation positive fish, and F3 homozygous positive larvae fish were obtained by PCR screening from F2 generation positive self-bred offspring.

The invention constructs pT2-krt8-IFN1 and pTol2-krt8-IFN1 vectors, and co-microinjects with mature transposase mRNA synthesized in vitro into fertilized eggs of fish (such as loach, catfishes, etc.), and designs and optimizes two pairs of specific primers for IFN1 (krt8-F1/HIS tag-R1 and krt8-F2/INF1-R5), and develops a stable genetically-transformed homozygous loach.

The expression vector constructed by the present invention contains a tag sequence, and a bioactive substance can be purified by using a tag antibody: (1) The impurities in the protein solution are removed by centrifugation or ultrafiltration technology, and the centrifugation process is kept at a low temperature to ensure that the bioactive substance is not degraded or the activity is reduced. (2) using a corresponding purification column (such as a nickel column) to purify the protein solution; (3) using ion exchange equipment to further purify the protein. Throughout the purification process, SDS-PAGE staining and Western blot methods can be used to verify the purification effect.

The present invention needs to select different activity test schemes for different types of biologically active substances: (1) for antiviral biologically active substances (such as IFN1, etc.), the antiviral activity can be detected by using a minimal cytopathic inhibition method, including PCR detection of the replication of the infected virus and the transcription and expression of key factors of its downstream signaling pathways; (2) at the living level, the bioactive substance purified from the mucus at the appropriate dose (such as 1 μg/g body weight) by intraperitoneal or intramuscular injection to introduce into the experimental animal infected with the virus, the number of deaths is continuously observed and recorded to determine the antiviral effect of the biologically active substance; for the antibacterial biologically active substance (such as antibacterial peptides), the intraperitoneal injection method is used to detect the antibacterial activity of the biologically active substance in the mucus.

The novel fish bioreactor for skin and mucous glands developed by the invention can efficiently express various biologically active substances. Application schemes include: (1) construction of GMP production workshops, closed production of bioreactors, collection of mucus, extraction, purification and packaging of biologically active substances to achieve large-scale commercial production of biologically active substances; (2) optimization of product production, packaging, transportation, storage and use of various technical links, formulate technical specifications for the safe production of skin and mucous gland bioreactors and the use of biologically active substances; (3) establish demonstration bases, organize professional training, and promote the application of biologically active substances.

These and other objectives, features, and advantages of the present invention will become apparent from the following detailed description, the accompanying drawings, and the appended claims.

### BRIEF DESCRIPTION OF THE DRAWINGS

FIG. 1 is a flow chart according to a preferred embodiment of the present invention.

FIG. 2 shows the skin-specific genes screened by RT-PCR and whole-mount in situ hybridization analysis; wherein FIG. 2A is a diagram showing RT-PCR detection of lgals2b, rblec, glant8 gene tissue expression. FIG. 2B is a diagram showing the WISH with lgals2b probes to detect the mRNA expression in loach at 96 hpf.

FIG. 3 is the protein difference between different tissues, wherein FIG. 3A is the mass spectrometry pre-treatment steps for different tissue protein samples; FIG. 3B is the Wayne diagram of the protein types identified by each tissue, including Muscle, Liver, Intestine, Gill and Mucus.

FIG. 4 is a transcriptional activity analysis diagram of the krt8 promoter in loach embryos.

FIG. 5 is diagram showing a test of the loach glant8 gene promoter activity.

FIG. 6 is a diagram showing the transgenic vector; wherein FIG. 6A is a diagram showing the transgenic vector pT2-krt8-IFN1; FIG. 6B is a diagram showing the transgenic vector pTol2-krt8-IFN1.

FIG. 7 is a diagram showing the detection of IFN1 signal peptide secretion using 293T cells; FIG. 7A is a schematic diagram of 293T cell culture and trans-fection. FIG. 7B is a diagram showing western blot analysis of 20 μL samples collected culture supernatant of each day.

FIG. 8 is a diagram showing the in vitro expression of grass carp IFN1; wherein FIG. 8A is a diagram showing the vector pCDNA-IFN1-His; FIG. 8B is a diagram showing expression of cIFN in vitro.

FIG. 9 is a diagram showing an analysis of the antiviral activity of grass carp IFN1; wherein FIG. 9A shows the growth of CIK cells after adding GCRV873 virus particles; (FIG. 9B) shows GCRV873-S5 which is a subunit of GCRV873 virus; FIG. 9C GCRV873-S6 is another subunit of GCRV873 virus; FIG. 9D IFR-9 is diagram showing a downstream factor of the IFN1 signaling pathway; FIG. 9E is a diagram showing STAT1 being a key signaling molecule in the IFN1 signaling pathway.

FIG. 10 is a diagram showing the preparation of microinjection samples; wherein FIG. 10A is an electrophoresis diagram after double digestion of plasmid pT2-krt8-IFN1 (4.22 k, 2.46 k) by Ade I and Xho I; FIG. 10B is an electrophoresis diagram after synthesis of capped mRNA. M is a DNA molecular weight marker.

FIG. 11 is the development route of genetically modified loach and screening of positive fish; wherein FIG. 11A is a diagram showing a strategy for generation of transgenic loach; FIG. 11B is a diagram showing primers designed on the trans-gene; FIG. 11C is a diagram showing sensitivity and specificity of seven primer sets were determined by PCR in a 25 μL volume containing 100 ng genomic DNA and transgenic plasmids pT2-krt8-IFN1 (0, 1, 5, 10, 20, 50 or 100 copies) as temples.

FIG. 12 is an analysis of the integration site of transgenic IFN1 gene in loach; wherein FIG. 12A is a diagram showing Southern blot analysis of transgenic IFN1 gene loach; FIG. 12B is a diagram showing Schematic diagram of integration of a transgenic vector into a genome; FIG. 12C is a g=diagram showing the electrophoresis of Genome walking. AD5 and AD1 are merging primers of the genome of loach. R1, R2, R3 and L1, L2, L3 are primers designed by transgenic plasmid.

FIG. 13 is a transcription and translation expression analysis of the transfected IFN1 gene; wherein FIG. 13A is diagram showing a transcript detection of IFN1; FIG. 13B is a diagram showing protein expression in vitro detected by Western blot.

FIG. 14 is a diagram shows the results of protein purification.

### DETAILED DESCRIPTION OF THE PREFERRED EMBODIMENT

In order to make the objectives, technical solutions, and advantages of the present invention clearer and clearer, the present invention is further described in detail below with reference to the examples. The equipment and reagents used in the examples and test examples can be obtained from commercial sources unless otherwise specified. The specific embodiments described here are only used to explain the present invention, and are not intended to limit the present invention.

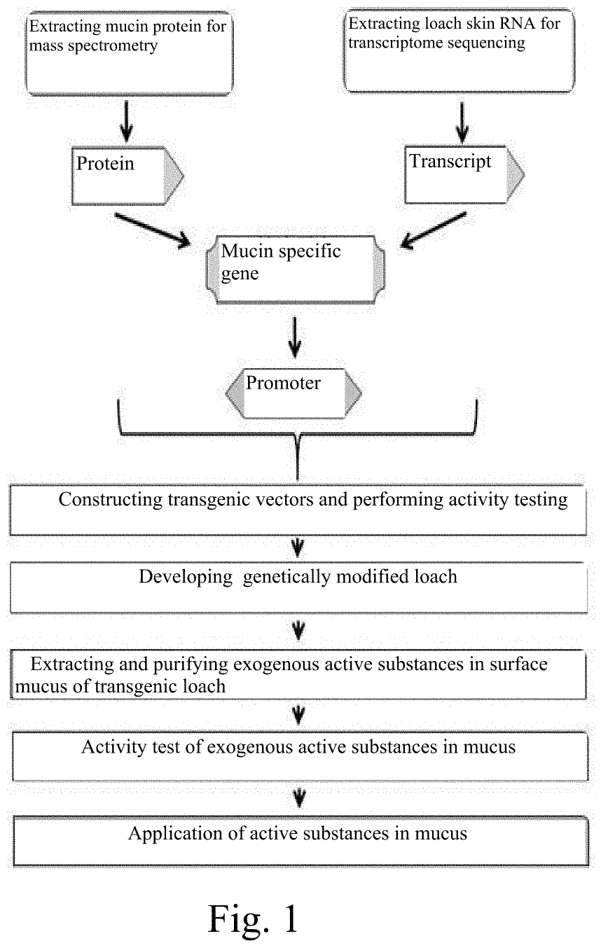

The invention discloses a method for preparing a fish skin mucous gland bioreactor and its application. The technical idea is shown in FIG. 1, which includes: firstly cloning and obtaining fish skin and mucous gland cell-specific promoters, as described in Example 1 below and implemented Example 3 uses three methods in combination with bioinformatics technology to search for and clone promoters of genes specifically expressed in skin and mucus gland cells. Thereafter, the activity of the specific promoter was detected by the technical scheme of Example 4. A transgenic expression vector capable of specifically expressing a biologically active substance was constructed by the technical solution of Example 5. In Example 6, the transgenic expression vector constructed in Example 5 is used to detect whether the bioactive substance produced has the ability to be secreted into mucus. In Example 7, the transgenic expression vector constructed in Example 5 was used to test whether the biologically active substance expressed in vitro has the corresponding activity. Example 8 shows construction of a transgenic fish containing a corresponding biologically active substance gene. Example 9 and Example 10 analyze the transgene integration site, transcription and translation expression in transgenic fish, respectively. Example 11 provides a method for purifying bioactive substances in skin mucous glands in transgenic fish. Example 12 provides a method for detecting the activity of bioactive substances in skin mucus in transgenic fish. Example 13 provides the application of bioactive substances in fish skin mucus.

The technical solution of the present invention is described in detail below with reference to specific examples. Except for special instructions, in each example, the molecular cloning experiments were performed under conventional conditions and with reference to Sambrook et al. (Second Edition, 1992, Science Press).

### Example 1: Identification of Skin-Specifically Expressed Genes by Genomics Technology, Acquisition of Fish Skin and Mucous Gland Cell-Specific Promoters

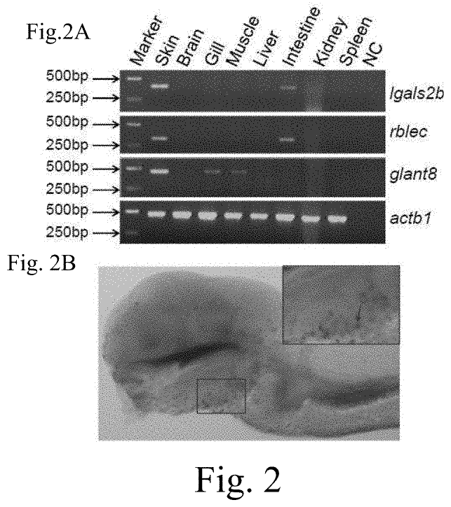

Total RNA from loach skin was extracted, a sequencing library was constructed, and transcriptome sequencing was performed using a high-throughput sequencing platform. The sequencing results were assembled by De novo using Velvet software, and a total of 40364 cDNA sequences were obtained. Through blasting genome database, comparison and analysis, the gene and cDNA sequences with high abundance expression in skin were obtained. Then, based on the protein cDNA sequence, PCR primers and RNA probes were designed. RT-PCR and whole-mount in situ hybridization analysis were performed. Among the genes that were highly expressed, lgals2b, rblec3, and glant8 were screened. Three genes that can be specifically expressed in the skin and intestine of loach were shown in FIG. 2. (A) RT-PCR detected the tissue distribution of genes. Loach lgals2b and rblec genes are specifically expressed in skin and intestine, glant8 is mainly expressed in skin, and weakly expressed in gills and muscles. (B) Expression pattern of loach glant8 gene. A whole-mount in situ hybridization analysis was performed with 96 hpf loach larvae after fertilization. Boxes show enlarged areas and arrows indicate skin mucus cells.

### Example 2: Identification of Mucus-Specific Proteins by Proteomics Techniques, Acquisition of Fish Skin and Mucous Gland Cell-Specific Promoters

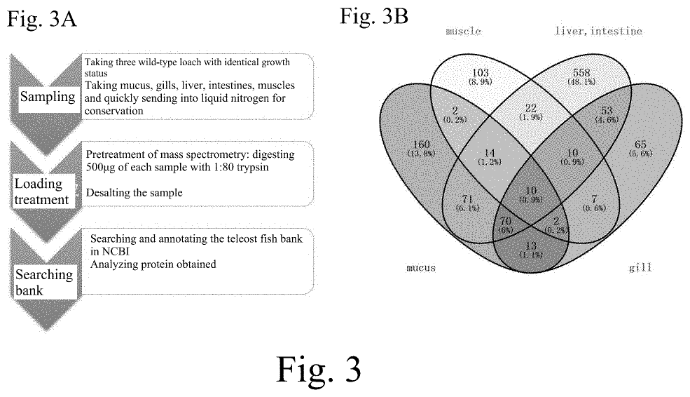

Extract different tissue samples and mucus from the gills, muscles, intestines, livers, etc. of the loach, and perform sample pretreatment on the proteins according to the proteomic mass spectrometry requirements. Because loach lacks genomics data, the present invention compares mass spectrometry data with the NCBI teleost database, analyzes samples from different tissues, and finds 160 specific proteins in loach slime samples. The results are shown in FIG. 3, wherein (A) is different mass specimen lading steps for tissue protein samples. (B) Venn diagram of the protein types identified by each tissue; Muscle, Liver, Intestine, Gill, Mucus. The GI number of the loach mucus-specific protein was converted into the UniProt KB format through the UniProt database, and a database search was performed to remove unknown and non-existent proteins in the database. Finally, 75 loach mucus-specific protein genes were obtained and shown one by one.

### Example 3: Searching Published Literature and Discovering Genes or Promoters Specifically Expressed in Fish Skin and Mucous Gland Cells

Test whether the existing krt8 promoter can drive the specific expression of target genes in loach skin.

1. Total RNA is extracted from 24-48 hpf zebrafish embryos. After crushing, total RNA is extracted according to the operating instructions of TRIZOL® Reagent. Take 1 μL RNA electrophoresis to check the quality of RNA and determine the RNA concentration. Take 4 μg of newly prepared RNA for reverse transcription, aliquot the remaining RNA and store at −80° C. until use.

2. Synthesis of the First Strand cDNA by Reverse Transcription

(1) Add the following reagents to the nuclease-free PCR tube in sequence: Total RNA, 5 μL (4 μg); Oligo-dT primer, 1 μL; Nuclease-free, 6 μL; total volume, 12 μL. (2) After mixing and centrifuging, put in a PCR machine and react at 70° C. for 5 min. (3) After standing on ice for 2 min, add the following reagents in sequence: 5× buffer, 4 μL; RNase Inhibitor, 1 μL; 10 mM dNTP mix, 2 μL. (4) Mix gently and centrifuge. Leave at 37° C. for 5 min (25° C. for 5 min if random primer is used). (5) Leave on ice for 2 min, add 1 μL of reverse transcriptase, mix and centrifuge. (6) PCR program: 42° C., 60 min; 70° C., 10 min (if random primer is used, the program is: 25° C., 10 min; 42° C., 60 min; 70° C. 10 min). (7) Stop the reaction on ice.

3. PCR Amplification of Krt8 Promoter Sequence

Vector NTI was used to design primers krt8-F and krt8-R (Table 1). The zebrafish cDNA was used as a template to obtain the DNA sequence of the krt8 promoter. The PCR system was: 10× Tag buffer, 2.5 μL; dNTP, 0.5 μL; krt8-F, 0.5 μL; krt8-R, 0.5 μL; cDNA, 1 μL; 2× Tag, 0.5 μL; ddH2O, 19.5 μL. The total volume is 25 μL.

PCR reaction conditions: pre-denaturation at 94° C. for 5 min, denaturation at 94° C. for 30 s, annealing at 58° C. for 30 s, 72° C. extension for 150 s, 30 cycles, and finally extension at 72° C. for 10 min. The resulting reaction product was electrophoresed on a 1% agarose gel. The target band was recovered according to the experimental method of the gel recovery kit (BioFlux). The recovered DNA band was sequenced and compared with the sequence in NCBI. The sequence of the krt8 promoter region obtained is shown in SEQ ID NO. 1.

4. TA clone ligation: Linearize plasmid pT2-HB (Perry Hackett, University of Minnesota, USA) with restriction enzymes Hind III and EcoR I to obtain a 3558-bp DNA fragment. The promoter sequence of krt8 obtained in the above reaction was double-digested with the restriction enzymes Hind III and EcoR I. Then two DNA fragments were ligated by TA to obtain pT2-krt8-6 #. The ligation system was: insert, 3 μL; linearized vector, 1 μL; T4 ligase, 1 μL; 10× buffer, 1 μL; PEG4000, 1 μL; ddH2O, 3 μL. The total volume is 10 μL. Ligation at 16° C. overnight. According to the above method, EGEP in pTME-Z48 (Generation of an Enhancer-Trapping Vector for Insertional Mutagenesis in Zebrafish, 2015) was cloned into pT2-krt8-6 # to obtain pT2-krt8-EGFP plasmid.

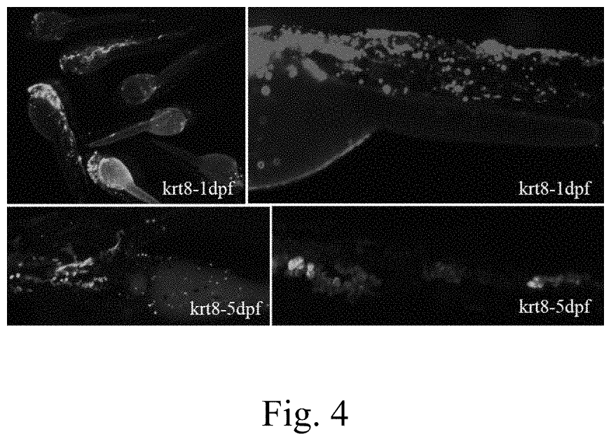

5. Fertilized eggs of loach at single cell stage was collected and the pT2-krt8-EGFP plasmid was microinjected to determine whether the krt8 promoter can drive specific expression of the target gene in loach skin. The results in FIG. 4 show that strong green fluorescence expression can be seen in the skin of loach embryos and fry at 1 and 5 days after fertilization, indicating that the krt8 promoter can drive EGFP expression well.

### Example 4: Activity Test of Fish Skin and Mucous Gland Cell-Specific Promoters

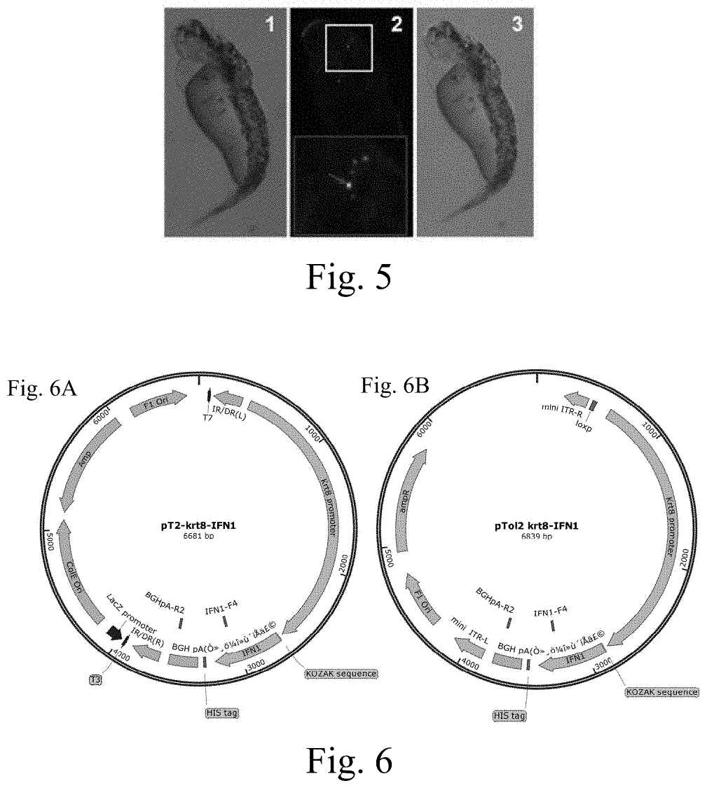

In the present invention, the full-length cDNAs of three loach skin-specific expression genes are cloned by using the RACE technology, and the upstream promoter sequence thereof is obtained by using genomic walking technology (Genome walking) to drive the expression of a green fluorescent protein (EGFP) reporter gene. The EGFP gene expression vector was microinjected into the single-celled embryo of loach, and the promoter of glant8 gene was found to drive the specific expression of EGFP in skin mucus cells. The results are shown in FIG. 5, where (1) white light, (2) EGFP, and (3) Overlay photos of white light and GFP; boxes indicate enlarged areas, and arrows indicate skin mucus cells.

### Example 5: Construction of a Transgenic Overexpression Vector

Molecular cloning techniques such as PCR, digestion, plasmid transformation and extraction were used to transform the pCDNA3.1 vector into pCDNA-IFN1-His. Then, pT2-krt8-GFP obtained in Example 3 was transformed into pT2-krt8-IFN1, as shown in FIG. 6A, pTol2-angptl3 (hu)-eGFP was transformed into pTol2-krt8-IFN1, as shown in FIG. 6B. The specific operations are as follows:

According to the method of Example 3, cDNA of grass carp kidney, intestine and spleen was obtained by reverse transcription. Primers (Table 1) were designed to amplify the cDNA of IFN1 gene by PCR. The PCR system was: 10× Tag buffer, 2 μL; dNTP, 0.4 μL; gcIFN1-F, 0.4 μL; gcIFN1-R, 0.4 μL; cDNA, 1 μL; 2× Tag, 2 μL; ddH2O, 19.8 μL. The total volume is 25 μL.

The pT2-krt8-6 # and pZero2-IFN-βGHPA-2 # were double digested with Ava I and BamHI, and then ligated to obtain pT2-krt8-IFN1. The digestion systems were: 1) 10× buffer, 5 μL; pT2-krt8-6 #, 12 μL; Bgl II, 1.25 μL; Xba I, 1.25 μL; ddH2O, 25.5 μL; 50 μL total volume. 2) 10× buffer, 5 μL; pZero2-IFN-βGHPA-2 #, 10 μL; Bgl II, 1.25 μL; Xba I, 1.25 μL; ddH2O, 27.5 μL; 50 μL total volume.

The obtained IFN1 cDNA sequence is shown in SEQ ID NO. 2.

Using a DNA gel recovery kit, the corresponding DNA fragments were recovered and ligated under the action of T4 ligase. The ligation system was: insert, 3 μL; linearized vector, 1 μL; T4 ligase, 1 μL; 10× buffer, 1 μL; PEG4000, 1 μL; ddH2O, 3 μL. The total volume is 10 μL. Ligation at 16° C. overnight.

### Example 6: Analytical Protocol for Signal Peptides of Biologically Active Substances

1. Recovery of 293T Cells

Before the cell operation involved in the present invention, the ultra-clean workbench needs to be sterilized by ultraviolet irradiation for 20 minutes, and the culture medium is preheated in a water bath at 37° C. or 28° C. for 20 minutes. Wipe all utensils and clean bench with alcohol cotton ball. Remove the 293T cells from the liquid nitrogen tank, quickly dissolve them at 37° C., and centrifuge at low speed (1200 rpm) for 5 min. Aspirate the medium from the cell tube (containing DMSO). Dissolve the bottom cells with 1 mL of fresh medium and suspend the cells. Aspirate the liquid and transfer to a 35 mm cell culture dish, add appropriate fresh medium, and culture in a 37° C. cell incubator (be sure to keep it steady when you put it in, otherwise it will easily form concentric circles).

2. Passaging of 293T Cells

When the growth density of 293T cells reaches more than 90%, aspirate the culture medium in the petri dish, add 1.5 mL of trypsin, and digest at 37° C. for 1 min. Observe that the cell morphology becomes round under the microscope, indicating that the digestion is complete, and trypsin is removed. Add 3 mL of culture medium to the petri dish, and slowly blow with a pipette to disperse the cells. Then divide the 3 mL of the cell-containing culture solution into 2 new culture dishes, add 2 mL of culture medium each, and slowly blow with a pipette to disperse the cells and culture under 37° C. and 5% CO₂ culture conditions.

3. Transfection of 293T Cells

When the cell density reaches 70-80%, the cells are transfected. The medium in the cell culture dish was replaced with serum-free medium, and starved at 37° C. for 1 h. The transfection complex was prepared. The plasmid and transfection reagent were prepared according to the ratio of 1 μg plasmid and 3 μL transfection reagent. The plasmids were the control blank plasmids pCDNA3.1 and pCDNA-IFN1-His obtained in Example 5, dilute with serum-free medium, mix well and incubate at room temperature for 20 min. Add the transfection complex dropwise to the petri dish and shake to mix. Cells were cultured at 37° C. and 5% CO₂. After 8 h of transfection, the cells were replaced with growth medium, cultured at 37° C. with 5% CO₂ for 24 h, then replaced with serum-free medium, and the culture was continued. 200 μL of the medium was collected every 24 h.

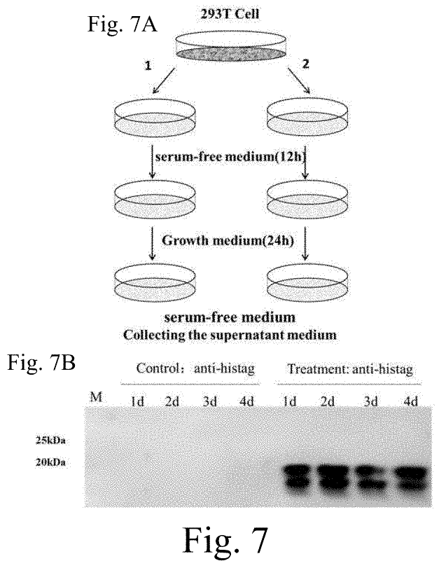

4. One of the target genes expressed by the present invention is the IFN1 gene of grass carp. To secrete IFN1 into mucus, a suitable signal peptide needs to be selected. Because grass carp's IFN1 has a signal peptide, the present invention first selects its own signal peptide. The control blank plasmids pCDNA3.1 and pCDNA-IFN1-His were transfected into 293T cells. After transfection, they were allowed to stand for 8 hours. After being replaced with growth medium, they were replaced with serum-free medium. The process is shown in FIG. 7A, where 1-Transfection of empty plasmid pCDNA3.1; 2-transfection of pCDNA-IFN1-His. In serum-free media collected on days 1-4, the expression of IFN1 could be detected by Western blot. The results are shown in FIG. 7B. These results indicate that IFN1 can be secreted into serum-free medium outside cultured cells under the guidance of grass carp IFN1's own signal peptide.

### Example 7: In Vitro Expression and Antiviral Activity Analysis of Grass Carp IFN1

1. Grass Carp IFN1 Expression In Vitro

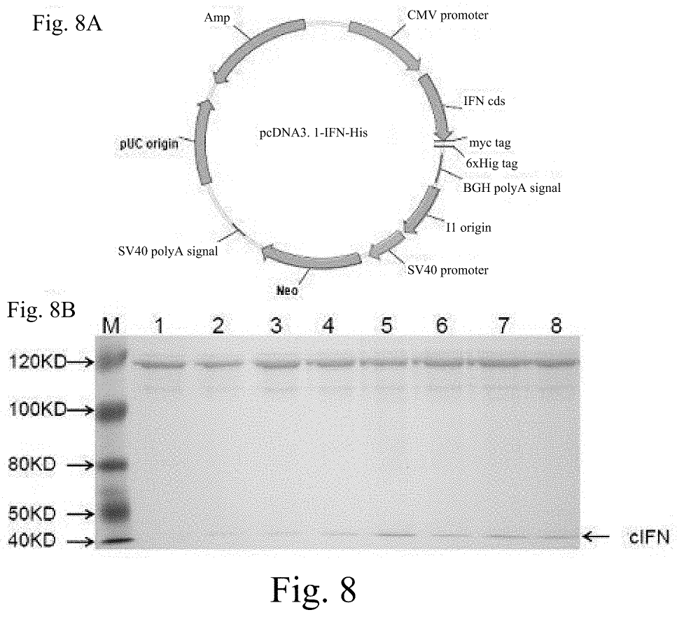

The cDNA of the IFN1 gene was cloned from grass carp tissue (see Example 5), and an overexpression vector was constructed and transfected into 293T cells. The specific method is described in Example 6. The culture medium was collected on day 0-7 and subjected to PAGE gel electrophoresis. In FIG. 8, coomassie brilliant blue staining was used to identify the expression of cIFN in vitro and successfully expressed grass carp IFN1.

2. Analysis of Antiviral Activity of Grass Carp IFN1

2.1. Transfection of Grass Carp Kidney Cell Line (CTK)

The CIK cell transfection step is the same as the above 293T cell transfection step, but CIK cells are more adherent than 293T cells, the enzymolysis time is about 6 minutes, and it is required to be performed in an incubator at a temperature of 28° C.

2.2. GCRV873 Infects CIK Cells

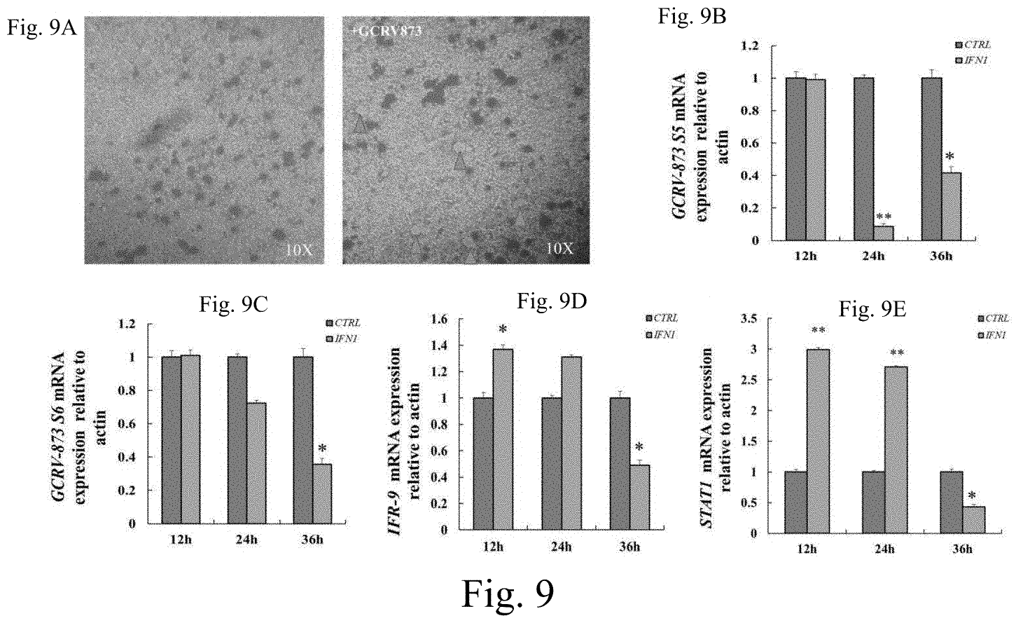

When the growth density of CIK cells in the culture dish reached more than 80%, a medium containing GCRV873 virus particles was added, and after 3 days of culturing at 28° C., it was found that cell plaques appeared in the culture dish (+GCRV873) added with GCRV873 virus particles, see FIG. 9A, indicating that the RNA virus particles of GCRV873 can cause apoptosis of CIK cells.

2.3. GCRV873 Virus Infection and Activity Detection

CIK cells were transfected with pCDNA3.1 and pCDNA-IFN1-His plasmids respectively, with pCDNA3.1 as the control group. After 6 hours, the medium containing GCRV873 virus particles was changed, and the cell growth was observed. Primers were designed to detect the mRNAs of the two subunits of GCRV873 (GCRV873-S5-F/R and GCRV873-S6-F/R, Table 1). See FIGS. 9B and 9C, and the transcriptional expression of IFN1-STAT1 downstream acting factors IFR-9 (IFR-9-F/R, Table 1) and STAT1 (STAT1-F/R, Table 1), see FIGS. 9D and 9E. The results showed that grass carp IFN1 expressed in vitro had antiviral activity.

TABLE 1
Primer sequences

| Primer name | Sequence (5′-3′) | Note |
|---|---|---|
| gcIFN1-F | CTCGAGATGAAAACTCAAATGTGGACG, See SEQ | Molecular cloning |
| | ID NO. 3 | |
| gcIFN1-R | CCTAGGAGCAGACAACCGTTACGAAC, See SEQ ID | Molecular cloning |
| | NO. 4 | |
| krt8-F | CCTTCCCTTCTAAGTCTGACG, See SEQ ID NO. 5 | Promoter cloning |
| krt8-R | GATGCCTGTGTCTTTGAGTTG, See SEQ ID NO. 6 | Promoter cloning |
| Krt8-F1 | CAGAGGGACTTTGACTCTCCTTTG, See SEQ ID NO. 7 | Primer optimization |
| HIS tag-R1 | ATGATGATGATGATGATGGTCG, See SEQ ID NO. 8 | Primer optimization |
| Krt8-F2 | GAATGCCTGTCCTCAAGTCTCAAG, See SEQ ID | Primer optimization |
| | NO. 9 | |
| INF1-R5 | CGTCCTGGAAATGACACCTTGG, See SEQ ID NO. 10 | Primer optimization |
| BGHpA-F1 | CGACTGTGCCTTCTAGTTGCC, See SEQ ID NO. 11 | Primer optimization |
| BGHpA-R2 | GCCTGCTATTGTCTTCCCAATC, See SEQ ID NO. 12 | Primer optimization |
| His tag-F1 | CGACCATCATCATCATCATCATTG, See SEQ ID | Primer optimization |
| | NO. 13 | |
| IFN1-F2 | CGATACAGGATGATAAGCAACGAG, See SEQ ID | Transcript analysis |
| | NO. 14 | |
| IFN1F4 | CAGCCATCACATAAGGAGTCC, See SEQ ID NO. 15 | Transcript analysis |
| IR-F1 | CTGTATCACAATTCCAGTGGGTC, See SEQ ID NO. 16 | Transcript analysis |
| krt8-R5 | GGCATTTAATAGCATTACGCAATCG, See SEQ ID | Transcript analysis |
| | NO. 17 | |
| STAT1-F | AGACCAGCAAGACGAATACGA, See SEQ ID NO. 18 | Protein activity analysis |
| STAT1-R | TGTTGACGGCACCTCCATT, See SEQ ID NO. 19 | Protein activity analysis |
| IRF-9-F | GCTGGACATCTCAGAACCTTAC, See SEQ ID NO. 20 | Protein activity analysis |
| IRF-9-R | CTCCTCCTGCTGCTCCTTAC, See SEQ ID NO. 21 | Protein activity analysis |
| GCRV873- | GTGGCACGGCTCTGCAAGTT, See SEQ ID NO. 22 | Protein activity analysis |
| S5-F | | |
| GCRV873- | CAACCGAGGCACCATCAACCAT, See SEQ ID NO. 23 | Protein activity analysis |
| s5-R | | |
| GCRV873- | TGCGACAACGGCTGCTTTGAT, See SEQ ID NO. 24 | Protein activity analysis |
| S6-F | | |
| GCRV873- | TTGCGGACAACCAACGGATGG, See SEQ ID NO. 25 | Protein activity analysis |
| s6-R | | |
| R1 | ATGTAAACTTCTGACCCACTGGGAATG, See SEQ | Chromosome walking |
| | ID NO. 26 | |
| R2 | TGGTGATCCTAACTGACCTAAGACAG, See SEQ ID | Chromosome walking |
| | NO. 27 | |
| R3 | CGACTTCAACTGAGTCGACCTCG, See SEQ ID NO. 28 | Chromosome walking |
| L1 | TCAGACTTAG AAGGGAAGGA AGC, See SEQ ID | Chromosome walking |
| | NO. 29 | |
| L2 | AGTAGATGTCC TAACTGACTT GCC, See SEQ ID | Chromosome walking |
| | NO. 30 | |
| L3 | ATAGTGAGTCGTA TTACGCGCGCT, See SEQ ID | Chromosome walking |
| | NO. 31 | |
| AD1 | TGWGNAGWANCASAGA, See SEQ ID NO. 32 | Chromosome walking |
| AD5 | STAGNATSGNGTNCAA, See SEQ ID NO. 33 | Chromosome walking |
| AD6 | WGCANGAWGNAGNATG, See SEQ ID NO. 34 | Chromosome walking |
| AD7 | NTCGTSGNATSTWGAA, See SEQ ID NO. 35 | Chromosome walking |

### Example 8: Preparation of Transgenic Fish

1. Synthesis of Mature mRNA In Vitro

(1) Linearization: Add the sample on ice, digest the vector pSBRNAX for linearization with Not I (the vector was donated by the Perry Hackett Laboratory of the University of Minnesota, USA), and the digestion reaction system: ddH2O, 90 μL; 10× buffer, 12 μL; pSBRNAX, 12 μL (6 μg); Not I (10 U/μL), 6 μL; total volume 120 μL. After adding each reaction component, mix well and divide into 12 tubes, and water bath at 37° C. for 16 h. (2) After electrophoretic tapping, use BioFluxDNA gel recovery kit to recover pSBRNAX linearized with Not I. See the instructions for the recovery procedure, and dissolve in DEPC water in the last step. (3) Transcription: The mature mRNA synthesis kit is used for transcription. The transcription system is as follows: 2×NTP/cap, 10 μL; 10×buffer, 2 μL; pSBRNAX linearized template, 6 μL; Enzyme mix, 2 μL; total volume 20 μL. After adding each reaction component, mix well, 37° C. water bath, 120 min. (4) Add 1 μL1 U/μL DnaseI, 37° C. for 30 min. (5) Add 30 μL Nucleare-free water and 30 μL LiCl, and precipitate at −20° C. for at least 30 min or −80° C. overnight. (6) Centrifuge at 12000 g for 15 min at 4° C.; wash with 70% DEPC in ethanol; centrifuge at 12000 g for 15 min at 4° C.; leave at room temperature for 5 min. (7) Dissolved in 10 μL DEPC water.

2. Linearized Transgenic Vector pT2-Krt8-IFN1

(1) Load on ice. Reaction system: ddH2O, 90 μL; 10×buffer G, 12 μL; pT2-krt8-IFN1, 12 μL (6 μg); Ade I (10 U/μL), 3 μL; Xho I (10 U/μL), 3 μL; total volume is 120 μL. After adding each reaction component, mix well, divide into 12 tubes, and water bath at 37° C. for 16 h. (2) After electrophoretic tapping, use BioFluxDNA gel recovery kit to recover pT2-krt8-IFN1 after double digestion with Ade I and Xho I. (3) Filling: ddH2O, 11 μL; recovered product, 15 μL; Pfu buffer, 2.5 μL; DNTP, 0.5 μL; Pfu, 1 μL; mix well, 72° C., PCR reaction for 20 min. (4) Recovered with BioFluxDNA gel recovery kit, dissolved in DEPC water, and used for microinjection after measuring the concentration.

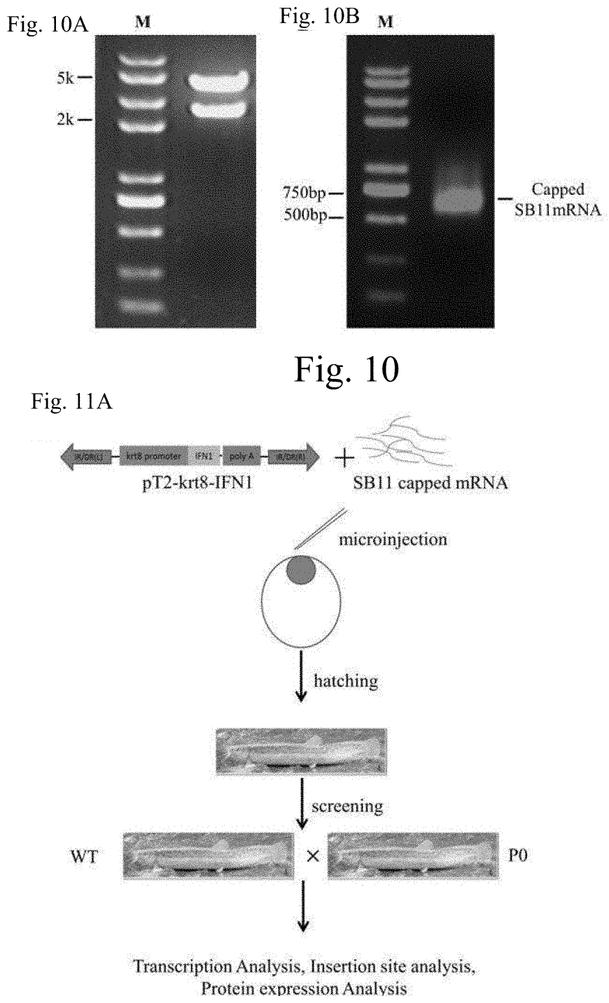

The results are shown in FIG. 10, in which (A) Ade I and Xho I double-digested the plasmid pT2-krt8-IFN1 (4.22 kb, 2.46 kb) after electrophoresis. (B) Electrophoresis image after synthesis of capped mRNA. M: DNA molecular weight marker.

3. Preparation of Loach Embryos

The sexually mature female and male loach are separated, placed in a large plastic box with aerated water, and oxygen is continuously supplied by a pump. The water temperature is controlled at about 25° C. and fed twice a day. At about 4:30 pm the day before breeding, the females were given the first aphrodisiac. Carp pituitary: After grinding, dissolve with 0.75% physiological saline, and inject an amount of half pituitary into each loach, and reduce the male fish by half. The second aphrodisiac interval was 6 hours. Both male and female were aphrodisiac. After 10 hours, gently squeeze the belly of the female bonito for inspection, and check every half an hour until the eggs can be laid.

4. Embryo microinjection

The entire operation process is guaranteed to be free of nuclease contamination. 50 ng/μL of the transposase capped mRNA is mixed with 25 ng/μL of the linearized gene expression vector pT2-krt8-IFN1 or pTol2-krt8-IFN1 and injected into single cells. At the stage of fertilized eggs of loach, the injection site is the junction of the blastoderm and yolk, and the injection volume is about 2 nL. The injected embryos were placed in a petri dish containing fish culture water and placed in a 28° C. incubator.

5. Positive Screening of Transgenic Loach

5.1 Extraction of the Caudal Fin Genome

Dispense 10 mL of the crude extract lysate containing PTK into 24 tubes of 1.5 mL centrifuge tubes, 400 μL each. Use surgical scissors to carefully remove a small amount of loach caudal fins (don't cut the bleeding). The surgical scissors need to be burned and cooled on an alcohol lamp before cutting the fins. After cutting the fish tail, place it in a 56° C. water bath shaker for 6 h, shake the centrifuge tube at regular intervals during the period, and speed up the cracking of the tail fin. When the lysate becomes clear, the tissue is completely lysed. Remove from a 56° C. water bath shaker, add 800 μL (pre-cooled at −20° C.) absolute ethanol to each tube to flocculate the genomic DNA. Centrifuge at 12000 rpm for 3 min. Remove the supernatant, wash the DNA with pre-chilled 75% ethanol, and centrifuge at 12,000 rpm for 2 min. Remove the supernatant, centrifuge at 12,000 rpm for 1 minute, and use a pipette to aspirate the residual liquid. Allow to air dry for 10 min at room temperature, and add 40 μL ddH2O to dissolve in 56° C. water bath for 30 min. Store at −20° C. for future use.

5.2 Use the method described above to extract the caudal fin genomic DNA, identify the positive fish by PCR, and the reaction system of transgenic positive fish PCR: ddH2O, 7.4 μL; 2×ES tag mix, 10 μL; forward primer (10 μM), 0.8 μL; Reverse primer (10 μM), 0.8 μL; caudal fin genomic DNA, 1 μL; total volume was 20 μL.

PCR reaction conditions: pre-denaturation at 94° C. for 5 min, denaturation at 94° C. for 30 s, annealing at 55° C. for 30 s, extension at 72° C. for 50 s, 30 cycles, and finally extension at 72° C. for 10 min. The resulting reaction product was electrophoresed on a 1% agarose gel.

The technical route and the results of PCR identification are shown in FIG. 11, in which (A) the development route of the transgenic loach. (B) Relative position of transgenic primer design. (C) Detection of PCR sensitivity and specificity of 7 pairs of primers. The PCR reaction system was a mixture of 25 μL, 100 ng loach genome and different copy gradients (0 copy, 1 copy, 5 copies, 10 copies, 20 copies, 50 copies, and 100 copies) of the transgenic plasmid pT2-krt8-IFN1 as a template. The gene transfer efficiency of the P0-F2 generation is shown in Table 2.

TABLE 2
PCR results of transgenic loach

| Transgenic | Total | Positive | Transgenic |
|---|---|---|---|
| generation | number | number | efficiency (%) |
| P0 | 260 | 9 | 3.46 |
| F1 | 528 | 35 | 6.62 |
| F2 | 95 | 23 | 24.2 |

The DNA fragment sequence obtained by PCR of positive fish is shown in SEQ ID NO. 1.

### Example 9: Analysis of Integration Sites of IFN1 Transgenic Fish

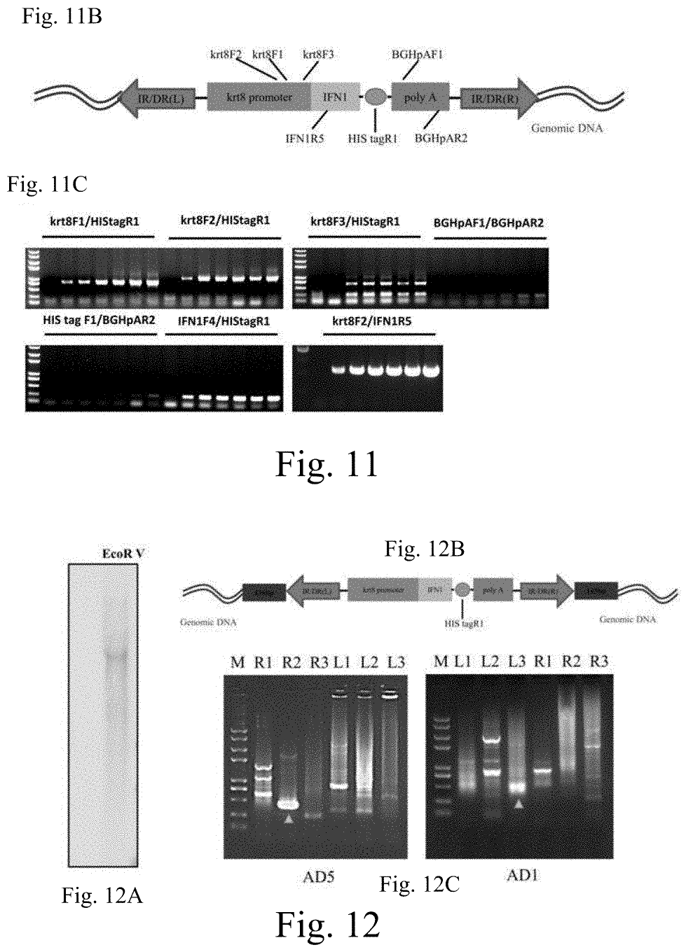

F1-positive individuals were selected, and Southern blotting and Genome walking techniques were used to analyze the integration site of the transgenic gene, and the insertion site of the transgenic gene was analyzed and verified (See FIG. 12). Because loach has no complete genome data, it is impossible to determine the chromosome and specific location of the inserted gene, and only the upstream and downstream DNA sequences of the insertion site can be obtained.

1. Southern Blot

1.1 Restriction of the Genome

The genome of the F1 transgenic loach embryo was extracted, and a total of 5 μg of the genome was digested with EcoR V in a 50 μL system. The digestion system was: 4 μL EcoR V, 21 μL genomic DNA (238 ng/μL), 5 μL buffer, 22 μL ddH2O. 50 μL total. Digest at 37° C. overnight. Every 24 hours, 2 μL of the digested product is electrophoresed to test the digestion efficiency of the genome. If the genome is digested to a uniform and dispersed state, the reaction is terminated.

1.2 Electrophoresis and Gel Processing

A 0.7% agarose gel was prepared, and the genomic digestion product was electrophoresed at a voltage of 20-40V until the bromophenol blue ran to the other end of the gel to terminate the electrophoresis. Soak the gel block in ethidium bromide buffer solution for 10 minutes, observe the electrophoresis, remove the excess part of the gel block, and mark the corner of the gel for incision. Rinse twice with ddH2O on a horizontal shaker for 10 min each. Rinse the denaturing buffer twice for 20 min each. Rinse ddH2O twice for 5 min each.

1.3 Transfer Film

(1) Place a platform larger than the gel on the disk, and lay 3 pieces of 3 mm filter paper on the platform. The two ends of the paper hang into the transfer buffer in the disk. Use a scalpel to cut a piece of nylon film with a large gel, soak the nylon film with distilled water for 5-10 minutes, and cut a corner to correspond to the glue. (2) Remove the gel from the water, turn it over so that its back is facing up, and place it in the center of the filter paper on the transfer platform to ensure that there are no air bubbles between the paper and the gel. Use plastic wrap to seal around the gel to prevent short-circuiting during the transfer process. (3) Place the nylon membrane on the gel to remove air bubbles. (4) Take 3 pieces of 3 mm filter paper of the same size as the gel, moisten them with 2×SSC, and place them on a nylon membrane. (5) Put a stack of absorbent paper (5-8 cm) on the filter paper, then press a glass plate and a weight of 500-750 g. (6) Transfer overnight. Replace the filter paper one or more times after the filter paper is wet. There must be enough transfer liquid in the tray to ensure continuous transfer work. (7) After the transfer, discard the paper towel, flip the nylon membrane and gel with the gel facing up, and mark the position of the sample well on the nylon membrane with a pencil. To estimate the DNA transfer efficiency, use 0.5 μg/L for the gel. Ethidium bromide was stained for 20-45 min, and the transfer was observed under ultraviolet light. (8) Rinse the nylon membrane at 6×SSC for 10 min to remove the agarose adhered to the membrane. (9) Take out the nylon membrane, drain the solution, place it on filter paper, and dry it at room temperature for more than 30 minutes. (10) Load the dried nylon membrane in the middle of the two filter papers, bake in a vacuum oven at 80° C. for 2-4 h, or cross-link with a UV lamp for 2 min to fix the DNA on the membrane.

1.4 Preparation of DIG-Labeled DNA Probes

We selected part of the gene sequence of the expression vector as the DNA probe template. The primer krt8-F1/IFN1-R 5 sequence is shown in the primer list 1. Digoxin-labeled DNA probes were obtained using digoxin-labeled primers based on random primer labeling techniques. Probe preparation method is described in the DIG High Prime DNA Labeling and Detection Starter Kit II manual.

1.5 Hybridization and Detection of Nylon Membrane

For hybridization and immunoassay procedures, refer to the instructions of DIG High Prime DNA Labeling and Detection Starter Kit II.

2. Chromosome Walking

According to the principles and steps provided by Takara's chromosome walking kit, the present invention has made a certain degree of optimization, which has greatly reduced the experimental cost and achieved good results. The enzyme in the kit was replaced with Takara Ex Tag, and experiments were performed with degenerate primers AD5, AD6, and AD7 (Table 1) designed with the primers in the kit.

The specific experimental procedure is as follows: The genomic DNA of loach was refined using the method described previously. Based on the known genomic sequence, three primers were designed in the same direction: R1, R2 and R3. The primer sequences are shown in Table 1. The principle of primer design is based on Takara chromosome walking kit.

Reaction system for the first round of PCR (using AD5 primers as an example): template DNA, 0.5 μL (50-500 ng); 10× Ex PCR Buffer, 2.5 μL; dNTP Mixture (2.5 mM each), 4 μL; AD5 (100 μM), 0.5 μL; R1 (10 μM), 0.5 μL; Takara Ex Taq (5 U/μL), 0.2 μL; ddH2O, 16.8 μL; total volume was 25 μL.

The reaction procedure is as follows: 1 cycle: 94° C., 1 min; 1 cycle: 98° C., 1 min; 5 cycles: 94° C., 30 s; 65° C., 1 min; 72° C., 2 min; 1 cycle: 94° C., 30 s; 25° C., 3 min; 72° C., 2 min; 15 large cycles: 94° C., 30 s; 65° C., 1 min; 72° C., 2 min; 94° C., 30 s; 65° C., 1 min; 72° C., 2 min; 94° C., 30 s; 44° C., 1 min; 72° C., 2 min; 1 cycle: 72° C., 10 min.

Reaction system for the second round of PCR: the first round of PCR products, 0.5 μL; 10× Ex PCR Buffer, 2.5 μL; dNTP Mixture (2.5 mM each), 4 μL; AD5 (100 μM), 0.5 μL; R2 (10 μM), 0.5 μL; TaKaRa Ex Taq (5 U/μL), 0.2 μL; ddH2O, 16.8 μL; total volume is 25 μL.

The reaction procedure is as follows: 15 large cycles: 94° C., 30 s; 65° C., 1 min; 72° C., 2 min; 94° C., 30 s; 65° C., 1 min; 72° C., 2 min; 94° C., 30 s; 44° C., 1 min; 72° C., 2 min; 1 cycle: 72° C., 10 min.

Reaction system of the third round of PCR: the second round of PCR products, 0.5 μL; 10× Ex PCR Buffer, 2.5 μL; dNTP Mixture (2.5 mM each), 4 μL; AD5 (100 μM), 0.5 μL; R3 (10 μM), 0.5 μL; TaKaRa Ex Taq (5 U/μL), 0.2 μL; ddH2O, 16.8 μL; total volume is 25 μL.

The reaction procedure is as follows: 15 large cycles: 94° C., 30 s; 65° C., 1 min; 72° C., 2 min; 94° C., 30 s; 65° C., 1 min; 72° C., 2 min; 94° C., 30 s; 44° C., 1 min; 72° C., 2 min; 1 cycle: 72° C., 10 min.

15 μL of the first and second rounds of PCR products and the third round of PCR products were all analyzed by agarose gel electrophoresis. The third round was about 100 bases smaller than the second round. The target band was cloned and sequenced.

The results are shown in FIG. 12, wherein (A) is a Southern blotting imprint. (B) is a pattern map of pT2-krt8-IFN1 in the genome. (C) is a chromosome walking electrophoresis. The R-terminus (AD5) of the sequencing sequence in FIG. 12C is shown in SEQ ID NO. 2, and the L-terminus (AD1) is shown in SEQ ID NO. 3.

### Example 10: Analysis of Transcription and Translation Expression of Transplanted IFN1 Gene

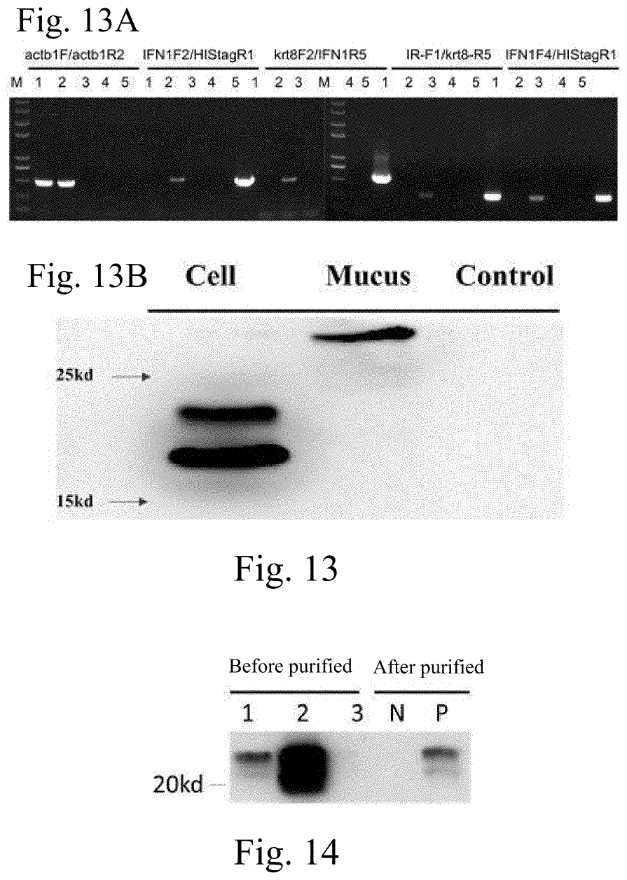

According to the TRIZOL RNA extraction method of molecular cloning, total RNA of F1 generation fish fins was extracted, and then reverse digestion with DNase, primers (IFN1-F2/HIStag-R1, krt8-F2/IFN1-R5, IR-F1/krt8-R5 and IFN1-F4/HIStag-R1, Table 1) were used to detect the presence of IFN1 gene transcripts in transgenic loach, the results are shown in FIG. 13A. Mucus protein was extracted, and the expression of IFN1 protein was detected using HIS antibody See FIG. 13B. The mucin protein was digested according to the ratio of trypsin: protein (1:80), and the protein was fully digested into peptides, and then detected by mass spectrometry. The mass spectrum results showed that the mucin protein contained the target protein IFN1.

### Example 11: Purification Protocol of Bioactive Substances in Skin Mucous Glands of Transgenic Loach

After transfecting 293T cells with pcNA-IFN1-His plasmid, a large amount of medium supernatant was collected, and then the interferon was purified using Novagen His•Bind resin. After the purification was completed, the purification effect was detected by Western blot. The results See FIG. 14, where 1, 2, and 3 represent the first, second, and third tubes collected in sequence after adding the elution buffer, respectively: N: negative control, P: positive control. The results show that the resin purification method is effective for purification of bioactive substances expressed in vitro or in skin mucous glands.

1. Protein Purification: Histones Carry 6×His-Tag. In the Present Invention, his Bind Resin from Novagen is Used to Purify Interferon.

(1) Thaw the cell culture medium collected in batches at room temperature, place on ice for 5-10 min, and add 8× bound buffer at a ratio of 7:1. (2) Invert the vial containing the resin upside down to resuspend the resin evenly. Take a 1.6 mL suspension with a wide-mouth pipette and add it to a pre-prepared empty column (the column volume is 0.8 mL after complete sedimentation). 3 mL of sterilized ultrapure water, blow flat, and allow the resin to settle naturally under the effect of gravity, about 30 min. (3) Open the piston. When the resin settles and the liquid level drops to the resin surface, clean, ionize, and equilibrate the column in the following order: 3 mL of sterilized ultrapure water; 4 mL of 1× ionization buffer; 3 mL 1× binding buffer. (4) When the binding buffer is quickly lowered onto the resin surface, cover the plunger and carefully add 10 mL of the culture medium. (5) Unscrew the plunger, thoroughly drain the medium from the column, and add 3 mL of 1× binding buffer to wash away unbound protein. (6) 10 mL of 1× rinsing buffer to wash away non-specifically bound proteins. (7) After the liquid has completely dried, add 3 mL of 1× elution buffer, stop the outlet with a stopper, blow it evenly with a gun, and let it stand. (8) After 5 min, unscrew the stopper, the first few drops of liquid are not needed, and collect the 3 mL elution buffer, that is, the purified recombinant protein, in three tubes in time order. (9) Nickel ions bound to the resin were stripped with 5 mL of stripping buffer. (10) The resin was stored in a peel buffer at 4° C.

2. Western Blot Detection:

(1) Gel making: Assemble the rubber fittings according to the instructions, and make sure that everything is dry and clean before assembling. The gel preparation kit was used to prepare 15% separation gel and 5% concentrated gel. (2) Gel running: Assemble the running gel and after preparing, put the gel in the electrophoresis instrument, add 1× SDS running buffer to completely submerge the gel and flood the bottom groove to ensure that there is no leakage. Add 15 μL of protein sample and 10 μL of pre-stained maker. Constant voltage 90 V, 1.5 h. (3) Transfer film The PVDF film is slightly smaller than the gel soaked in methanol. Simultaneously immerse the filter paper and fiber pad in transfer buffer. Assemble them in the order of fiber mat, filter paper, gel membrane, filter paper, and fiber mat, put them into the transfer membrane tank, fill the transfer membrane buffer solution, and put the stirrer in the transfer membrane tank. The film trough is placed in a large basin filled with ice cubes. Connect the power in a refrigerator at 4° C. and transfer 300 mA constant current for 2 h. (4) Place the membrane in blocking solution after transferring the membrane, and block the membrane for at least 1 h at room temperature on a horizontal shaker. (5) Incubate the membrane in a primary antibody (His•Tag® Antibody HRP) at 4° C. overnight. (6) Recover primary antibody and wash TBST 6 times for 10 min each time. (7) Mix the same volumes of Western chemiluminescence HRP substrates: Peroxide Solution and Luminol Reagent before color development and photography. (8) Drop the chemiluminescence mixture evenly on the film and take a picture.

### Example 12: Activity Test Scheme of Bioactive Substances in Skin Mucus of Transgenic Loach

1. Cell Level

According to the method of Example 11, the mucus on the surface of the loach was collected. Centrifuge the mucus and filter to remove impurities such as cell debris from the mucus. PMSF and cocktail were added at a rate of 1% to prevent protein degradation. Mucus protein samples were purified through a nickel column to obtain higher concentration protein samples containing IFN1, and the protein concentration was measured using a BCA kit. The grass carp IFN1 protein samples of different concentrations extracted from the mucus were added to the CIK cell culture medium infected with GCRV873 virus. The method refers to Example 7, where the grass carp IFN1 standard is a positive control and the wild mucus sample is a negative control According to the method of FIG. 9, the growth status of CIK cells in each group, the mRNA expression level of GCRV873 subunit, and the mRNA expression of IFN1 signaling pathway and downstream key factors were detected.

The results showed that IFN1 protein samples in mucus could reduce GCRV873's replication ability and its expression level. At the same time, it can activate the expression of its downstream acting factors and improve its antiviral ability.

2. In Vivo Level

Purified grass carp IFN1 was added to the grass carp kidney cell line CIK medium infected with GCHV873, and the activity of grass carp IFN1 was measured by the method described in FIG. 9. In vivo level, according to a dose of 1 μg/g body weight, the purified grass carp IFN1 was injected into the Gobiocypris rarus by intraperitoneal injection, and the Gobiocypris rarus injected with the grass carp IFN1 was infected with GCHV873 virus particles. The number of deaths was continuously observed and recorded, and calculated. Relative antiviral efficiency.

The results showed that the death rate of the experimental group (Gobiocypris rarus tadpoles infected with GCHV873 virus particles and treated with grass carp IFN1) was lower than that of the control group (Gobiocypris rarus tadpoles infected with GCHV873 virus particles).

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method for preparing a fish skin mucous gland bioreactor, comprising steps of:
S1: obtaining specific expression promoters of fish skin and mucous gland cell;
S2: constructing a transgenic expression vector capable of specifically expressing the target biologically active substance, wherein the transgene expression vector comprises the specific expression promoters obtained in step S1; and
S3: digesting the transgenic expression vector obtained in step S2 and inject into the fertilized eggs of the fish to obtain transgenic fish.

2. The method for preparing the fish skin mucous gland bioreactor, as recited in claim 1, wherein the step S1 obtaining specific expression promoters of fish skin and mucous gland cell comprises: using transcriptomics, comparing and analyzing gene expression profiles of different tissues or cells of fish, identifying genes specifically expressed in fish skin and mucus gland cells, and searching for specific expression promoters of genes; or using proteomics techniques, comparing and analyzing protein expression profiles of different tissues or cells, identifying genes specifically expressed in fish skin and mucous gland cells, and searching for promoters of specifically expressed genes; or searching published literature to find genes or promoters specifically expressed in fish skin and mucous gland cells.

3. The method for preparing the fish skin mucous gland bioreactor, as recited in claim 2, wherein searching for the promoter of a specifically expressed gene is to use a genomic sequence alignment method or a chromosome walking technique, combined with biological information analysis to find the promoter sequence upstream of the target gene.

4. The method for preparing the fish skin mucous gland bioreactor, as recited in claim 1, wherein the specific expression promoter of fish skin and mucous gland cells in step S1 is krt8 or lgals2b or rblec3 or glant8 or other mucous-specific promoters.

5. The method for preparing the fish skin mucous gland bioreactor, as recited in claim 1, wherein the target biologically active substance in step S2 is selected from any one of polypeptides, proteins, enzymes, vaccines, antibodies, or feed additives.

6. The method for preparing the fish skin mucous gland bioreactor, as recited in claim 1, wherein the step S3 is followed by a step of purifying the bioactive substance secreted by the skin mucous glands.

7. The method for preparing the fish skin mucous gland bioreactor, as recited in claim 6, wherein the step of purifying the biologically active substance includes purifying the biologically active substance with a resin.

8. The method for preparing the fish skin mucous gland bioreactor, as recited in claim 1, wherein the fish are all mucus-secreting fish, including loach or catfish.

9. A method for industrially producing biological active substances comprising introducing the method for preparing the fish skin mucous gland bioreactor as recited in claim 1.

10. A method for preparing polypeptides, proteins, enzymes, vaccines, antibodies, or feed additives comprising introducing the method for preparing the fish skin mucous gland bioreactor as recited in claim 1.
